(19) United States
(12) Patent Application Publication
(10) Pub. No.: US 2005/0209218 A1
(43) Pub. Date: Sep. 22, 2005

(54) Methods and compositions for the treatment of psychiatric conditions

(76) Inventors: Laurence R. Meyerson, Carlsbad, CA (US); Gregory T. Went, Mill Valley, CA (US); Timothy J. Fultz, Pleasant Hill, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/058,118

(22) Filed: Feb. 14, 2005

Related U.S. Application Data
(60) Provisional application No. 60/544,838, filed on Feb. 13, 2004.

(51) Int. Cl. A61K 31/551; A61K 31/506; A61K 31/137; A61K 31/343

(52) U.S. Cl. 514/220; 514/252.15; 514/317; 514/469; 514/649

(57) ABSTRACT

This invention relates to methods and compositions for treating psychiatric conditions, such as depression.

### RELATED APPLICATION

This application claims priority to U.S. Ser. No. 60/544,838, filed Feb. 13, 2004. The contents of this application are incorporated herein by reference in its entirety.

### FIELD OF THE INVENTION

This invention relates to methods and compositions for treating psychiatric conditions, such as depression.

### BACKGROUND OF THE INVENTION

Recurrent mood disorders can have devastating long-term effects, and the cost of these illnesses in terms of human suffering, productivity and health care is enormous. It is now recognized that, for many patients, the long-term outcome is often much less favorable than previously thought, with incomplete interepisode recovery, and a progressive decline in overall functioning observed. Indeed, according to the Global Burden of Disease Study, mood disorders are among the leading causes of disability worldwide, and are likely to represent an increasingly greater health, societal, and economic problem in the coming years.

Many antidepressants are currently available for the treatment of acute depression. Until a few decades ago, tricyclic antidepressants (TCAs) were the only drugs available for the treatment of depression. A number of new drugs followed in rapid succession, among them the selective serotonin reuptake inhibitors (SSRIs) and serotonin/norepinepherine reuptake inhibitors (SNRIs) which are now widely used. Although options for pharmacologic treatment for depression have grown seemingly exponentially over the past several decades, the current armamentarium of antidepressants continues to have limitations of both efficacy and tolerability.

Thus, there is a clear need to develop novel and improved therapeutics for the treatment of major depression, especially refractory depression, bipolar depression, and the degeneration associated with depression.

### SUMMARY OF THE INVENTION

In general, the present invention provides methods and compositions for treating CNS-related conditions, such as psychiatric disorders and pain, by administering to a subject in need thereof a combination that includes an NMDA receptor antagonist and an anti-depressant drug (ADD). The administration of the combinations described herein results in the alleviation and prevention of symptoms associated with or arising from CNS-related conditions including, for example, including but not limited to depression, bipolar depression, anxiety headache, pain, neuropathies, cereborischemia, dementias, movement disorders, multiple sclerosis, and other psychiatric disorders. The active pharmaceutical agents may be administered to the patient in a manner that reduces the variability of the ratio of the concentrations of the active agents over a period of time, thereby maximizing the therapeutic benefit while minimizing the side effects. The present invention differs from prior studies by providing novel combinations as well as formulations of combinations directed to dose optimization or release modification to reduce adverse effects associated with each agent.

The NMDA receptor antagonist, the ADD, or both agents may be provided in a controlled or extended release form with or without an immediate release component in order to maximize the therapeutic benefit of each, while reducing unwanted side effects associated with each. When these drugs are provided in an oral form without the benefit of controlled or extended release components, they are released and transported into the body fluids over a period of minutes to several hours.

The NMDA receptor antagonist, the ADD, or both agents may be administered in an amount similar to that typically administered to subjects. Optionally, the amount of the NMDA receptor antagonist, the ADD, or both agents may be administered in an amount greater than or less than the amount that is typically administered to subjects. For example, the amount of memantine required to positively affect the patient response (inclusive of adverse effects) may be 2.5-80 mg per day rather than the typical 10-20 mg per day administered without the improved formulation described herein. A higher dose amount of the NMDA receptor antagonist in the present invention may be employed for conditions such as non-neuropathic pain whereas a lower dose of the NMDA receptor antagonist may be sufficient when combined with the ADD to achieve a therapeutic effect in the patient. Optionally, lower or reduced amounts of both the NMDA receptor antagonist and the ADD are employed in a unit dose relative to the amount of each agent when administered as a monotherapy.

As used herein, “C” refers to the concentration of an active pharmaceutical ingredient in a biological sample, such as a patient sample (e.g. blood, serum, and cerebrospinal fluid). The concentration of the drug in the biological may be determined by any standard assay method known in the art. The term “Cmax” refers to the maximum concentration reached by a given dose of drug in a biological sample. The term “Cmean” refers to the average concentration of the drug in the sample over time. Cmax and Cmean may be further defined to refer to specific time periods relative to administration of the drug. The time required to reach the maximal concentration (“Cmax”) in a particular patient sample type is referred to as the “Tmax”. The agents of the combination are administered in formulations that reduce the variability of the ratio of the concentrations of the active agents over a period of time, thereby maximizing the therapeutic benefit while minimizing the side effects.

If desired, the dosage form is provided in a non-dose escalating, twice per day or once per day form. In such cases, the concentration ramp (or Tmax effect) may be reduced so that the change in concentration as a function of time (“dC/dT”) is altered to reduce or eliminate the need to dose escalate the drug. A reduction in dC/dT may be accomplished, for example, by increasing the Tmax in a relatively proportional manner. Accordingly, a two-fold increase in the Tmax value may be reduce dC/dT by approximately a factor of 2. Thus, the NMDA receptor antagonist may be provided so that it is released at a dC/dT that is significantly reduced over an immediate release (so called IR) dosage form, with an associated delay in the Tmax. The pharmaceutical composition may be formulated to provide a shift in Tmax by 24 hours, 16 hours, 8 hours, 4 hours, 2 hours, or at least 1 hour. The associated reduction in dC/dT may be by a factor of approximately 0.05, 0.10, 0.25, 0.5 or at least 0.8. In certain embodiments, this is accomplished-by releasing less than 30%, 50%, 75%, 90%, or 95% of the NMDA receptor antagonist, the ADD, or both into the circulatory or neural system within one hour of such administration.

The ratio of the concentrations of two agents in a combination is referred to as the “Cratio”, which may fluctuate as the combination of drugs is released, transported into the circulatory system or CNS, metabolized, and eliminated. An objective of the present invention is to stabilize the Cratio for the combinations described herein. Beneficially, the variation in the Cratio (termed “Cratio, var”) should be as low as possible.

The present invention therefore features formulations of combinations directed to dose optimization or release modification to reduce adverse effects associated with separate administration of each agent. The combination of the NMDA receptor antagonist and the ADD may result in an additive or synergistic response, as described below.

Accordingly, in one aspect, the invention provides a pharmaceutical composition that includes an NMDA receptor antagonist, a second agent that is an anti-depressant drug (ADD), and, optionally, a pharmaceutically acceptable carrier. In some embodiments, at least one of the NMDA receptor antagonist or the second agent is provided in an extended release dosage form.

In another aspect, the invention features a method of preventing or treating a CNS-related condition by administering to a subject in need thereof a therapeutically effective amount of a combination comprising an NMDA receptor antagonist and a second agent that is an ADD. In some embodiments, at least one of the NMDA receptor antagonist or the second agent in the combination is provided in an extended release dosage form.

If desired, the NMDA receptor antagonist is released into a subject sample at a slower rate than observed for an immediate release (IR) formulation of the same quantity of the antagonist, wherein the release rate is measured as the dC/dT over a defined period within the period of 0 to Tmax for the IR formulation and the dC/dT rate is less than about 80% of the rate for the IR formulation. In some embodiments, the dC/dT rate is less than about 60%, 50%, 40%, 30%, 20%.or 10% of the rate for the IR formulation. Similarly, the ADD may also be released into a patient sample at a slower rate than observed for an IR formulation of the same quantity wherein the release rate is measured as the dC/dT over a defined period within the period of 0 to Tmax for the IR formulation and the dC/dT rate is less than about 80%, 60%, 50%, 40%, 30%, 20%, or 10%, of the rate for the IR formulation. In all foregoing aspects of the invention, if desired, at least 50%, 90%, 95%, or essentially all of the NMDA receptor antagonist in the pharmaceutical composition may be provided in a controlled release dosage form. In some embodiments, at least 99% of the NMDA receptor antagonist remains in the extended dosage form one hour following introduction of the pharmaceutical composition into a subject. The NMDA receptor antagonist may have a Cₘₐₓ/Cₘₑₐₙ of approximately 1.6, 1.5, 1.4, 1.3 or less, approximately 2 hours to at least 8, 12, 16, 24 hours after the NMDA receptor antagonist is introduced into a subject.

In all foregoing aspects of the invention, the second agent may also be provided in a controlled release dosage form. Thus, at least 50%, 60%, 70%, 80%, 90%, 95%, or essentially all of the AED may be provided as a controlled release formulation. If provided as such, the second agent has a Cₘₐₓ/Cₘₑₐₙ of approximately 1.6, 1.5, 1.4, 1.3 or less, approximately 2 hours to at least 6, 8, 12, 16, 24 hours after the second agent is introduced into a subject.

Optionally, the Cratio.var of the NMDA receptor antagonist, the AED, or both agents is less than 100%, e.g., less than 70%, 50%, 30%, 20%, or 10% after the agent(s) have reached steady state conditions or during the first 24 hours post-administration. In some embodiments, the Cratio.var is less than about 90% (e.g., less than about 75% or 50%) of that for IR administration of the same active pharmaceutical ingredients over the first 4, 6, 8, or 12 hours after administration.

The CNS-related condition that may be treated according to the present invention may be psychiatric disorders, (e.g., seizure., panic syndrome, general anxiety disorder, phobic syndromes of all types, mania, anxiety, manic depressive illness, hypomania, unipolar depression, depression, bipolar depression, stress disorders, PTSD, somatoform disorders, personality disorders, psychosis, and schizophrenia), and pain (e.g., acute pain, chronic pain, chronic neuropathic pain).

The combinations of the invention are also useful for the treatment and prevention of other disorders including headaches, cerebrovascular disease, motor neuron diseases, dementias, neurodegenerative diseases, strokes, movement disorders, ataxic syndromes, disorders of the sympathetic nervous system, cranial nerve disorders, myelopethies, traumatic brain and spinal cord injury, radiation brian injury, multiple sclerosis, post-menengitis syndrome, prion diseases, myelities, radiculitis, neuropathies, pain syndromes, axonic brain damage, encephalopathies, chronic fatigue syndrome, psychiatric disorders, and drug dependence.

In all foregoing aspects of the invention, the NMDA receptor antagonist may be an aminoadamantine derivative memantine (1-amino-3,5-dimethyladamantane), rimantadine (1-(1-aminoethyl)adamantane), or amantadine (1-amino-adamantane). The second agent may be a GABA transmaminase inhibitor, GABA re(uptake) inhibitor, carbonic anhydrase inhibitor, benzodiazepine, or sodium channel inhibitor. Alternatively, the second agent may be an anti-depressive agent that includes, for example, agents that block serotonin reuptake (SSRIs), block both serotonin and norepinepherine (SNRIs), act on dopamine receptors or block dopamine reuptake (TCAs, others). Exemplary anti-depressants drugs are the SSRIs (e.g., fluoxetine/PROZAC™, citalopram and escitalopram/CELEXA™ and LEXAPRO™, sertraline/ZOLOFT™, paroxetine/PAXIL™), SNRIs (e.g., duloxetine/CYMBALTA™, and venlafaxine/EFFEXOR™), TCAs (e.g., desipramine/NORPRAMIN™, imipramine/TOFRANIL™, cloimipramine/ANAFRANIL™, nortrytptline/PAMELOR™, and amitriptyline/ELAVIL™), bupropion/WELLBUTRIN™, and buspirone/BUSPAR™. Thus, the NMDA receptor antagonist may be memantine while the second agent may be fluoxetine, escitalopram, citalopram, duloxetine, or paroxetine.

The NMDA receptor antagonist, the second agent, or both agents are formulated for oral, parenteral, rectal, buccal, transdermal patch, transnasal , topical, subtopical transepithelial, subdermal, or inhalation delivery. Thus, the agents described herein formulated as a suspension, capsule, tablet, suppository, lotion, patch, or device (e.g., a subdermally implantable delivery device or an inhalation pump). If desired, the NMDA antagonist and the ADD may be admixed in a single composition. Alternatively, the two agents are delivered in separate formulations sequentially, or within one hour, two hours, three hours, six hours, 12 hours, or 24 hours of each other. If administered separately, the two agents may be administered by the same or different routes of administration three times a day, twice a day, once a day, or even once every two days.

Optionally, the NMDA receptor antagonist and the second agent are provided in a unit dosage form.

If desired, the amount of the NMDA receptor antagonist in the pharmaceutical composition is less than the amount of NMDA receptor antagonist required in a unit dose to obtain the same therapeutic effect for treating CNS-related condition when the NMDA receptor antagonist is administered in the absence of the second agent. Alternatively, the amount of the second agent in the pharmaceutical composition is less than the amount of the second agent required in a unit dose to obtain the same therapeutic effect for treating CNS-related condition when the second agent is administered in the absence of the NMDA receptor antagonist. Optionally, the NMDA receptor antagonist is present in the pharmaceutical composition at a dose that would be toxic to a human subject if the NMDA receptor antagonist were administered to the subject in the absence of the second agent. If desired, the second agent is present in the pharmaceutical composition at a dose that would be toxic to a human subject if the second agent were administered to the subject in the absence of the second agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting. All parts and percentages are by weight unless otherwise specified.

### BRIEF DESCRIPTION OF THE FIGURES

FIG. 1 is a graph showing that controlled release of the NMDA receptor antagonist results in a reduction in dC/dt.

FIG. 2A is a series of graphs showing the API concentrations over 24 hrs and 10 days for IR administration. Memantine is provided at 10 mg bid (Tmax 3hr, T½ 60 hr) and duloxetine is provided at 60 mg qd (Tmax 6hr, T½ 12 hr).

FIG. 2B is a series of graphs showing API concentrations over first 24 hours and 10 days for CR Formulation 1. Memantine is provided at 25 mg qd (Tmax 12hr, T½ 60 hr) while. duloxetine is provided at 60 mg qd (Tmax 12hr, T½ 12 hr).

FIG. 2C is a graph showing the ratio of duloxetine to Memantine concentrations for IR Administration and CR Formulation 1.

FIG. 2D is a graph showing the ratio of duloxetine to Memantine concentrations for IR Administration and CR Formulation 2.

FIGS. 3A-3F are graphs showing the PK profile release and Cratios of memantine and escitalopram as IR and CR formulations for example 6.

FIGS. 4A-4C are graphs showing the PK profile release and Cratios of memantine and escitalopram as IR and Patch formulations for example 7.

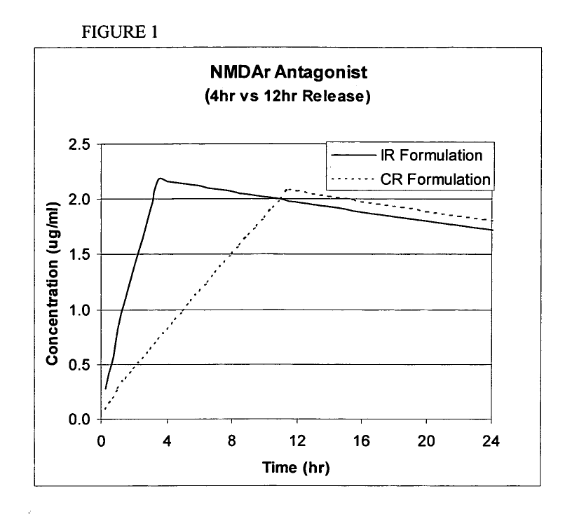

### DETAILED DESCRIPTION OF THE INVENTION

The present invention provides methods and compositions for treating or preventing CNS-related conditions, including psychiatric disorders (e.g., panic syndrome, general anxiety disorder, phobic syndromes of all types, mania, manic depressive illness, hypomania, unipolar depression, depression, stress disorders, PTSD, somatoform disorders, personality disorders, psychosis, and schizophrenia), and drug dependence (e.g., alcohol, psychostimulants (eg, crack, cocaine, speed, meth), opioids, and nicotine), epilepsy, headache, acute pain, chronic pain, neuropathies, cereborischemia, dementias, movement disorders, and multiple sclerosis. The combination includes a first component that is an NMDA receptor antagonist and a second component that is an anti-depressant drug (ADD). The combination is administered such that symptoms are alleviated or prevented, or alternatively, such that progression of the CNS-related condition is reduced. Desirably, either of these two agents, or even both agents, is formulated for extended release, thereby providing a concentration and optimal concentration ratio over a desired time period that is high enough to be therapeutically effective but low enough to avoid adverse events associated with excessive levels of either component in the subject.

NMDA Receptor Antagonists

Any NMDA receptor antagonist can be used in the methods and compositions of the invention, particularly those that are non-toxic when used in the combination of the invention. The term “nontoxic” is used in a relative sense and is intended to designate any substance that has been approved by the United States Food and Drug Administration (“FDA”) for administration to humans or, in keeping with established regulatory criteria and practice, is susceptible to approval by the FDA or similar regulatory agency for any country for administration to humans or animals.

The NMDA receptor antagonist may be an amino-adamantane compound including, for example, memantine (1-amino-3,5-dimethyladamantane), rimantadine (1-(1-aminoethyl)adamantane), amantadine (1-amino-adamantane), as well as pharmaceutically acceptable salts thereof. Memantine is described, for example, in U.S. Pat. Nos. 3,391,142, 5,891,885, 5,919,826, and 6,187,338. Amantadine is described, for example, in U.S. Pat. Nos. 3,152,180, 5,891,885, 5,919,826, and 6,187,338. Additional aminoadamantane compounds are described, for example, in U.S. Pat. Nos. 4,346,112, 5,061,703, 5,334,618, 6,444,702, 6,620,845, and 6,662,845. All of these patents are hereby incorporated by reference.

Further NMDA receptor antagonists that may be employed include, for example, ketamine, eliprodil, ifenprodil, dizocilpine, remacemide, iamotrigine, riluzole, aptiganel, phencyclidine, flupirtine, celfotel, felbamate, neramexane, spermine, spermidine, levemopamil, dextromethorphan ((+)-3-hydroxy-N-methylmorphinan) and its metabolite, dextrorphan ((+)-3-hydroxy-N-methylmorphinan), neramexane a pharmaceutically acceptable salt or ester thereof, or a metabolic precursor of any of the foregoing.

The NMDA receptor antagonist may be provided so that it is released at a dC/dT that is significantly reduced over an instant release (so called IR) dosage form, with an associated delay in the Tmax. The pharmaceutical composition may be formulated to provide a shift in Tmax by 24 hours, 16 hours, 8 hours, 4 hours, 2 hours, or at least 1 hour. The associated reduction in dC/dT may be by a factor of approximately 0.05, 0.10, 0.25, 0.5 or at least 0.8. In addition, the NMDA receptor antagonist may be provided such that it is released at rate resulting in a Cₘₐₓ/Cₘₑₐₙ of approximately 1.6, 1.5, 1.4, 1.3 or less for approximately 2 hours to at least 8, 12, 16, 24 hours after the NMDA receptor antagonist is introduced into a subject. The pharmaceutical composition may be formulated to provide memantine in an amount ranging between 1 and 80 mg/day, 5 and 40 mg/day, or 10 and 20 mg/day; amantadine in an amount ranging between 25 and 500 mg/day, 25 and 300 mg/day, or 100 and 300 mg/day; dextromethorphan in an amount ranging between 1-5000 mg/day, 1-1000 mg/day, and 100-800 mg/day, or 200-500 mg/day. Pediatric doses will typically be lower than those determined for adults. Representative dosing can be found in the PDR by anyone skilled in the art.

Table 1 shows exemplary the pharmacokinetic properties (e.g., Tmax and T½) for memantine, amantadine, and rimantadine.

TABLE 1
Pharmacokinetics and Tox in humans
for selected NMDAr antagonists

| | Human | | | | Dose |
|---|---|---|---|---|---|
| | PK (t½) | Tmax | Normal | | Dependent |
| Compound | in hrs | in hrs | Dose | | Tox |
| Memantine | 60 | 3 | 10-20 | mg/day, | Dose escalation |
| | | | starting at | | required, |
| | | | 5 | mg | hallucination |
| Amantadine | 15 | 3 | 100-300 | mg/day | Hallucination |
| Rimantadine | 25 | 6 | 100-200 | mg/day | Insomnia |

Anti-Depressant Drugs (ADDs)

Suitable anti-depressive agents include, for example, agents that block serotonin reuptake (SSRIs), block both serotonin and norepinepherine (SNRIs), act on dopamine receptors or block dopamine reuptake (TCAs, others). Exemplary anti-depressants drugs are SSRIs (e.g., fluoxetine/PROZAC™, citalopram and escitalopram/CELEXA™ and LEXAPRO™, sertraline/ZOLOFT™, paroxetine/PAXIL™), SNRIs (e.g., duloxetine/CYMBALTA™m, and venlafaxine/EFFEXOR™), TCAs (e.g., desipramine/NORPRAMIN™, imipramine/TOFRANIL™, cloimipramine/ANAFRANIL™, nortrytptline/PAMELOR™, and amitriptyline/ELAVIL™), bupropion/WELLBUTRIN™, and buspirone/BUSPAR™. Normal therapeutic doses can be found in the Physician desk reference (PDR), and are reflected below.

TABLE 2
Pharmacokinetics and Tox in humans for selected antidepressants

| | | | | | Main Dose |
|---|---|---|---|---|---|
| | Human PK | Tmax | Normal | | Dependent |
| Compound | T½ (hrs) | (hrs) | Dose | | Adverse Event |
| NORPRAMIN/ | 22 | 3-6 | 100-200 | mg/day | Hypotension, urinary |
| Desipramine | | | | | retention, QTC |
| LEXAPRO/ | 30 | 5 | 10-20 | mg/day | Sexual dys |
| Escitalopram | | | | | |
| PAXIL/ | 21 | 5 | 20-50 | mg/day | Sexual dys |
| Paroxetine | | | | | |
| CYMBALTA/ | 12 | 6 | 40-60 | mg/day | Dizziness |
| Duloxetine | | | | | |
| EFFEXOR/ | 5 parent, 11 | 2 parent/3 | 150-250 | mg/day | nausea, constipation, |
| Venlafaxine | for ODV | for ODV | | | anorexia, vomiting, |
| | | | | | somnolence, |
| BUSPAR/ | 7 | 1 | 20-30 | mg/day | Drowsiness, dizziness |
| Buspirone | | | | | |
| WELLBUTRIN/ | 14 | 2 | 200-300 | mg/day | Anorexia, constipation, |
| Bupropion | | | | | seizures (Bold Warning) |

In addition to the specific combinations disclosed herein, combinations made of a first NMDAr antagonist and an ADD may be identified by testing the ability of a test combination of a selected NMDAr antagonist and one or more ADD to lessen the symptoms of a CNS-related disorder. Preferred combinations are those in which a lower therapeutically effective amount of the NMDA receptor antagonist and/or ADD is present relative to the same amount of the NMDA receptor antagonist and/or ADD required to obtain the same anti-depressant effect when each agent is tested separately.

The amounts and ratios of the NMDA receptor antagonist and the ADD are conveniently varied to maximize the therapeutic benefit and minimize the toxic or safety concerns. The NMDA receptor antagonist may range between 20% and 200% of its normal effective dose and the ADD may range between 20% to 200% of its normal effective dose. The precise ratio may vary according to the condition being treated. In one example, the amount of memantine ranges between 2.5 and 40 mg per day and the amount of duloxetine ranges between 10 and 60 mg/day.

In addition to the specific combinations disclosed herein, combinations made of an NMDA receptor antagonist such as an aminoadamantane compound and an ADD may be identified by testing the ability of a test combination to lessen the symptoms of a CNS-related disorder (see Examples 1 and 2).

For a specified range a physician or other appropriate health professional will typically determine the best dosage for a given patient, according to his sex, age, weight, pathological state and other parameters. In some cases, it may be necessary to use dosages outside of the ranges stated in pharmaceutical packaging insert to treat a subject. Those cases will be apparent to the prescribing physician or veterinarian.

In some embodiments, the combinations of the invention achieve therapeutic levels while minimizing debilitating side-effects that are usually associated with immediate release formulations. Furthermore, as a result of the delay in the time to obtain peak plasma level and the potentially extended period of time at the therapeutically effective plasma level, the dosage frequency may be reduced to, for example, once or twice daily dosage, thereby improving patient compliance and adherence.

Accordingly, the combination of the invention allows the NMDA receptor antagonist and the ADD to be administered in a combination that improves efficacy and avoids undesirable side effects of both drugs. For example, side effects including psychosis and cognitive deficits associated with the administration of NMDA receptor antagonists may be lessened in severity and frequency through the use of controlled-release methods that shift the Tmax to longer times, thereby reducing the dC/dT of the drug. Reducing the dC/dT of the drug not only increases Tmax, but also reduces the drug concentration at Tmax and reduces the Cmax/Cmean ratio providing a more constant amount of drug to the subject being treated over a given period of time and reducing adverse events associated with dosing. Similarly, side effects associated with the use of ADDs may be reduced in severity and frequency through controlled release methods as well.

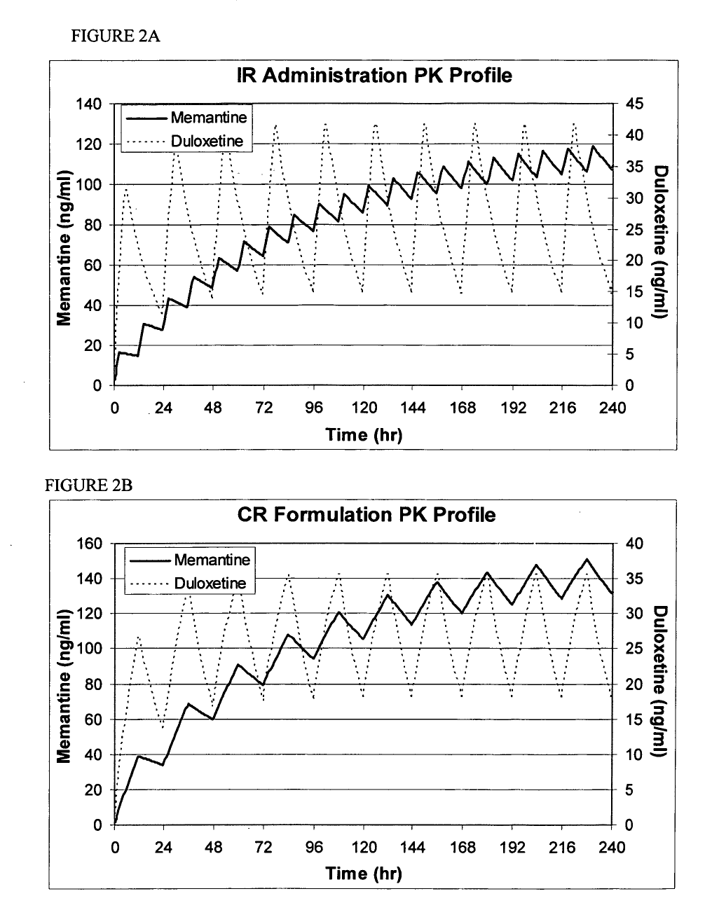

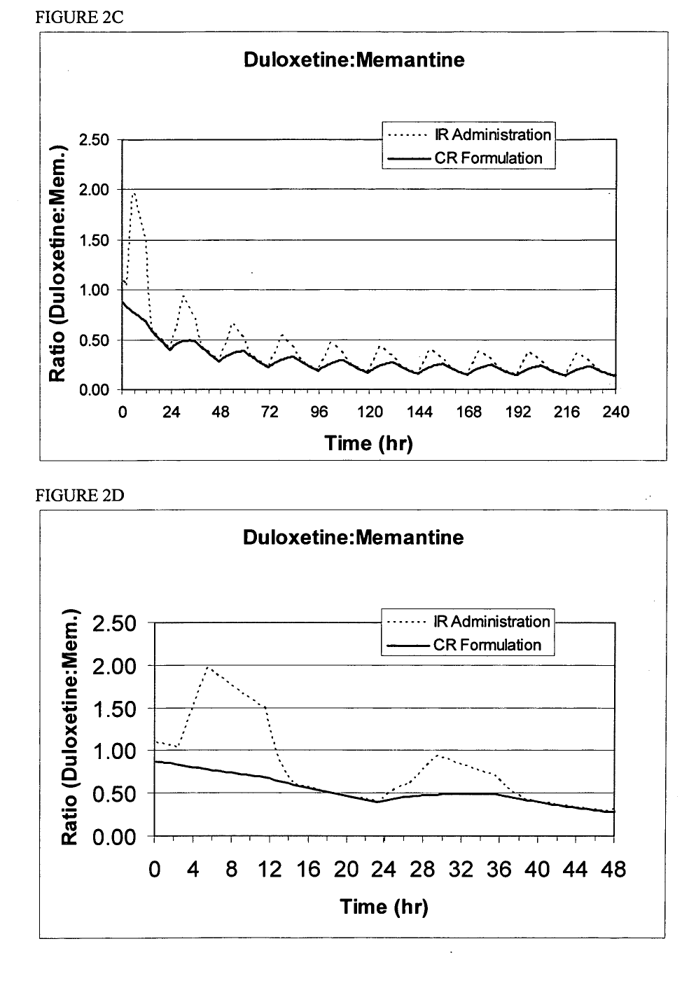

In certain embodiments, the combinations provide additive effects. Additivity is achieved by combining the active agents without requiring controlled release technologies. In other embodiments, particularly when the pharmacokinetic profiles of the combined active pharmaceutical ingredients are dissimilar, controlled release formulations optimize the pharmacokinetics of the active pharmaceutical agents to reduce the variability of the Cratio over time. Reduction of Cratio variability over a defined time period enables a concerted effect for the agents over that time, maximizing the effectiveness of the combination. The Cratio variability (“Cratio.var”) is defined as the standard deviation of a series of Cratios taken over a given period of time divided by the mean of those Cratios multiplied by 100%. As shown in FIGS. 2A-2D and in Table 3, the Cratio for the controlled release formulation is more consistent than for the IR administration of the same drug over any significant time period, including shortly after administration and at steady state. The data included in that figure are summarized in the table below:

TABLE 3
Memantine and Duloxetine Cratio and Cratio, var
Data in Immediate Release (IR) Administration
and Controlled Release (CR) Formulation

| | Time Period: | | Time period | |
|---|---|---|---|---|
| | 22-24 hrs | | 192-240 hours | |
| | IR | CR | IR | CR |
| Cratio range | 0.40-1.98 | 0.39-0.84 | 0.14-0.38 | 0.14-0.24 |
| Cratio mean | 1.04 | 0.62 | 0.24 | 0.19 |
| Cratio Std. Dev. | 0.57 | 0.14 | 0.07 | 0.03 |
| Cratio. var (%) | 55% | 23% | 30% | 16% |

Modes of Administration

The combination of the invention may be administered in either a local or systemic manner or in a depot or sustained release fashion. In a preferred embodiment, the NMDA receptor antagonist, the ADD, or both agents may be formulated to provide controlled, extended release (as described herein). For example, a pharmaceutical composition that provides controlled release of the NMDA receptor antagonist, the ADD, or both may be prepared by combining the desired agent or agents with one or more additional ingredients that, when administered to a subject, causes the respective agent or agents to be released at a targeted rate for a specified period of time. These agents may be delivered preferably in an oral, transdermal or intranasal form.

The two components are preferably administered in a manner that provides the desired effect from the first and second components in the combination. Optionally, the first and second agents are admixed into a single formulation before they are introduced into a subject. The combination may be conveniently sub-divided in unit doses containing appropriate quantities of the first and second agents. The unit dosage form may be, for example, a capsule or tablet itself or it can be an appropriate number of such compositions in package form. The quantity of the active ingredients in the unit dosage forms may be varied or adjusted according to the particular need of the condition being treated.

Alternatively, the NMDA receptor antagonist and the ADD of the combination may not be mixed until after they are introduced into the subject. Thus, the term “combination” encompasses embodiments where the NMDA receptor antagonist and the ADD are provided in-separate formulations and are administered sequentially. For example, the NMDA receptor antagonist and the ADD may be administered to the subject separately within 2 days, 1 day, 18 hours, 12 hours, one hour, a half hour, 15 minutes, or less of each other. Each agent may be provided in multiple, single capsules or tablets that are administered separately to the subject. Alternatively, the NMDA receptor antagonist and the ADD are separated from each other in a pharmaceutical composition such that they are not mixed until after the pharmaceutical composition has been introduced into the subject. The mixing may occur just prior to administration to the subject or well in advance of administering the combination to the subject.

If desired, the NMDA receptor antagonist and the ADD may be administered to the subject in association with other therapeutic modalities, e.g., drug, surgical, or other interventional treatment regimens. Where the combination includes a non-drug treatment, the non-drug treatment may be conducted at any suitable time so long as a beneficial effect from the co-action of the combination and the other therapeutic modalities is achieved. For example, in appropriate cases, the beneficial effect is still achieved when the non-drug treatment is temporally removed from the administration of the therapeutic agents, perhaps by days or even weeks.

Formulations for Specific Routes of Administration

Combinations can be provided as pharmaceutical compositions that are optimized for particular types of delivery. For example, pharmaceutical compositions for oral delivery are formulated using pharmaceutically acceptable carriers that are well known in the art. The carriers enable the agents in the combination to be formulated, for example, as a tablet, pill, capsule, solution, suspension, sustained release formulation; powder, liquid or gel for oral ingestion by the subject.

Alternatively, the compositions of the present invention may be administered transdermally via a number of strategies, including those described in U.S. Pat. Nos. 5,186,938, 6,183,770, 4,861,800 and WO 89/09051. Providing the drugs of the combination in the form of patches is particularly useful given that these agents have relatively high skin fluxes.

Pharmaceutical compositions containing the NMDA receptor antagonist and/or second agent of the combination may also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage can be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

In some embodiments, for example, the composition may be delivered intranasally to the. cribriform plate rather than by inhalation to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The combination may optionally be formulated for delivery in a vessel that provides for continuous long-term delivery, e.g., for delivery up to 30 days, 60 days, 90 days, 180 days, or one year. For example the vessel can be provided in a biocompatible material such as titanium. Long-term delivery formulations are particularly useful in subjects with chronic conditions, for assuring improved patient compliance, and for enhancing the stability of the combinations. Formulations for continuous long-term delivery are provided in, e.g., U.S. Pat. Nos. 6,797,283; 6,764, 697; 6,635,268, and 6,648,083.

If desired, the components may be provided in a kit. The kit can additionally include instructions for using the kit. In some embodiments, the kit includes in one or more containers the NMDA receptor antagonist and, separately, in one or more containers, the ADD. In other embodiments, the kit provides a combination with the NMDA receptor antagonist and the ADD mixed in one or more containers. The kits include a therapeutically effective dose of an agent for treating dementia-related conditions.

The NMDA receptor antagonist, the ADD or both agents may be provided in a controlled, extended release form. In one example, at least 50%, 90%, 95%, 96%, 97%, 98%, 99%, or even in excess of 99% of the NMDA receptor antagonist is provided in an extended release dosage form. A release profile, i.e., the extent of release of the NMDA receptor antagonist or the ADD over a desired time, may be conveniently determined for a given time by calculating the Cₘₐₓ/Cₘₑₐₙ for a desired time range to achieve a given acute or chronic steady state serum concentration profile. Thus, upon the administration to a subject (e.g., a mammal such as a human), the NMDA receptor antagonist has a Cmax_{/C}ₘₑₐₙ of approximately 1.6, 1.5, 1.4, 1,3 or less for approximately 2 hours to at least 8, 12, 16, 24 hours after the NMDA receptor antagonist is introduced into a subject. If desired, the release of the NMDA receptor antagonist may be monophasic or multiphasic (e.g., biphasic). Moreover, the ADD may be formulated as an extended release composition, having a Cₘₐₓ/Cₘₑₐₙ of approximately 1.6, 1.5, 1.4, 1,3 or less for approximately 2 hours to at least 8, 12, 16, 24 hours after the NMDA receptor antagonist is introduced into a subject. One of ordinary skill in the art can prepare combinations with a desired release profile using the NMDA receptor antagonists and the ADD and formulation methods known in the art or described below.

As shown in Tables 1 and 2, the pharmacokinetic properties of both of the drug classes vary from about 3 hours to more than 60 hours. Thus, one aspect of this invention is to select suitable formulations to achieve nearly constant concentration profiles over an extended period (preferably from 8 to 24 hours) thereby maintaining both components in a constant ratio and concentration for optimal therapeutic benefits for both acute and chronic administration. Preferred Cratio.var values are less than about 100%, 70%, 50%, 30%, 20%, 10%. Preferred Cratio.var values may be less than about 10%, 20%, 30%, 50%, 75%, or 90% of those for IR administration of the same active pharmaceutical ingredients over the first 4, 6, 8, 12 hours after administration.

Formulations that deliver this constant, measurable profile also allow one to achieve a monotonic ascent from an acute ratio to a desired chronic ratio for drugs with widely varying elimination half-lives. Compositions of this type and methods of treating patients with these compositions are embodiments of the invention. Numerous ways exist for achieving the desired release profiles, as described below.

Suitable methods for preparing combinations in which the first component, second component, or both components are provided in extended release-formulations include those described in U.S. Pat. No. 4,606,909 (hereby incorporated by reference). This reference describes a controlled release multiple unit formulation in which a multiplicity of individually coated or microencapsulated units are made available upon disintegration of the formulation (e.g., pill or tablet) in the stomach of the animal (see, for example, column 3, line 26 through column 5, line 10 and column 6, line 29 through column 9, line 16). Each of these individually coated or microencapsulated units contains cross-sectionally substantially homogenous cores containing particles of a sparingly soluble active substance, the cores being coated with a coating that is substantially resistant to gastric conditions but which is erodable under the conditions prevailing in the small intestine.

The combination may alternatively be formulated using the methods disclosed in U.S. Pat. No. 4,769,027, for example. Accordingly, extended release formulations involve prills of pharmaceutically acceptable material (e.g., sugar/starch, salts, and waxes) may be coated with a water permeable polymeric matrix containing an NMDA receptor antagonist and next overcoated with a water-permeable film containing dispersed within it a water soluble particulate pore forming material.

One or both components of the combination may additionally be prepared as described in U.S. Pat. No. 4,897,268, involving a biocompatible, biodegradable microcapsule delivery system. Thus, the NMDA receptor antagonist may be formulated as a composition containing a blend of free-flowing spherical particles obtained by individually microencapsulating quantities of memantine, for example, in different copolymer excipients which biodegrade at different rates, therefore releasing memantine into the circulation at a predetermined rates. A quantity of these particles may be of such a copolymer excipient that the core active ingredient is released quickly after administration, and thereby delivers the active ingredient for an initial period. A second quantity of the particles is of such type excipient that delivery of the encapsulated ingredient begins as the first quantity's delivery begins to decline. A third quantity of ingredient may be encapsulated with a still different excipient which results in delivery beginning as the delivery of the second quantity beings to decline. The rate of delivery may be altered, for example, by varying the lactide/glycolide ratio in a poly(D,L-lactide-co-glycolide) encapsulation. Other polymers that may be used include polyacetal polymers, polyorthoesters, polyesteramides, polycaprolactone and copolymers thereof, polycarbonates, polyhydroxybutyrate and copolymers thereof, polymaleamides, copolyaxalates and polysaccharides.

Alternatively, the combination may be prepared as described in U.S. Pat. No. 5,395,626 features a multilayered controlled release pharmaceutical dosage form. The dosage form contains a plurality of coated particles wherein each has multiple layers about a core containing an NMDA receptor antagonist and/or the ADD whereby the drug containing core and at least one other layer of drug active is overcoated with a controlled release barrier layer therefore providing at least two controlled releasing layers of a water soluble drug from the multilayered coated particle.

In some embodiments, the first component and second component of the combination described herein are provided within a single or separate pharmaceutical compositions. “Pharmaceutically or Pharmacologically Acceptable” includes molecular entities and compositions that do not produce an adverse, allergic or other untoward reaction when administered to an animal, or a human, as appropriate. “Pharmaceutically Acceptable Carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. “Pharmaceutically Acceptable Salts” include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The preparation of pharmaceutical or pharmacological compositions are known to those of skill in the art in light of the present disclosure. General techniques for formulation and administration are found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants and aerosols are examples of such formulations.

By way of example, extended release oral formulation can be prepared using additional methods known in the art. For example, a suitable extended release form of the either active pharmaceutical ingredient or both may be a matrix tablet composition. Suitable matrix forming materials include, for example, waxes (e.g., carnauba, bees wax, paraffin wax, ceresine, shellac wax, fatty acids, and fatty alcohols), oils, hardened oils or fats (e.g., hardened rapeseed oil, castor oil, beef tallow, palm dil, and soya bean oil), and polymers (e.g., hydroxypropyl cellulose, polyvinylpyrrolidone, hydroxypropyl methyl cellulose, and polyethylene glycol). Other suitable matrix tabletting materials are microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. Optionally, the finished tablet may be coated or uncoated.

The coating composition typically contains an insoluble matrix polymer (approximately 15-85% by weight of the coating composition) and a water soluble material (e.g., approximately 15-85% by weight of the coating composition). Optionally an enteric polymer (approximately 1 to 99% by weight of the coating composition) may be used or included. Suitable water soluble materials include polymers such as polyethylene glycol, hydroxypropyl cellulose, hydroxypropyl methyl cellulose, polyvinylpyrrolidone, polyvinyl alcohol, and monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups.

The coating composition may be plasticised according to the properties of the coating blend such as the glass transition temperature of the main component or mixture of components or the solvent used for applying the coating compositions. Suitable plasticisers may be added from 0 to 50% by weight of the coating composition and include, for example, diethyl phthalate, citrate esters, polyethylene glycol, glycerol, acetylated glycerides, acetylated citrate esters, dibutylsebacate, and castor oil. If desired, the coating composition may include a filler. The amount of the filler may be 1% to approximately 99% by weight based on the total weight of the coating composition and may be an insoluble material such as silicon dioxide, titanium dioxide, talc, kaolin, alumina, starch, powdered cellulose, MCC, or polacrilin potassium.

The coating composition may be applied as a solution or latex in organic solvents or aqueous solvents or mixtures thereof. If solutions are applied, the solvent may be present in amounts from approximate by 25-99% by weight based on the total weight of dissolved solids. Suitable solvents are water, lower alcohol, lower chlorinated hydrocarbons, ketones, or mixtures thereof. If latexes are applied, the solvent is present in amounts from approximately 25-97% by weight based on the quantity of polymeric material in the latex. The solvent may be predominantly water.

The pharmaceutical composition described herein may also include a carrier such as a solvent, dispersion media, coatings, antibacterial and antifungal agents, isotonic and absorption delaying agents. The use of such media and agents for pharmaceutically active substances is well known in the art. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition may also contain liquids, such as water, saline, glycerol, and ethanol, as well as substances such as wetting agents, emulsifying agents, or pH buffering agents. Liposomes, such as those described in U.S. Pat. No. 5,422,120, WO 95/13796, WO 91/14445, or EP 524,968 B1, may also be used as a carrier.

Additional methods for making controlled release formulations are described in, e.g., U.S. Pat. Nos. 5,422,123, 5,601,845, 5,912,013, and 6,194,000, all of which are hereby incorporated by reference.

Preparation for delivery in a transdermal patch can be performed using methods also known in the art, including those described generally in, e.g., U.S. Pat. Nos. 5,186,938 and 6,183,770, 4,861,800, and 4,284,444. A patch is a particularly useful embodiment in this case owing to absorption problems with many ADDs. Patches can be made to control the release of skin-permeable active ingredients over a 12 hour, 24 hour, 3 day, and 7 day period. In one example, a 2-fold daily excess of an NMDA receptor antagonist is placed in a non-volatile fluid along with an ADD. Given the amount of the agents employed herein, a preferred release will be from 12 to 72 hours.

Transdermal preparations of this form will contain from 1% to 50% active ingredients. The compositions of the invention are provided in the form of a viscous, non-volatile liquid. Preferably, both members of the combination will have a skin penetration rate of at least 10⁻⁹ mole/cm²/hour. At least 5% of the active material will flux through the skin within a 24 hour period. The penetration through skin of specific formulations may be measures by standard methods in the art (for example, Franz et al., J. Invest. Derm. 64:194-195 (1975)).

In some embodiments, for example, the composition may be delivered intranasally to the brain rather than by inhalation to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs.

Preparation of a pharmaceutical composition for delivery in a subdermally implantable device can be performed using methods known in the art, such as those described in, e.g., U.S. Pat. Nos. 3,992,518; 5,660,848; and 5,756,115.

Indications Suitable for Treatment with the Combination

Any subject having or at risk of having a CNS-related disorder, such as psychiatric disorders (e.g., panic syndrome, general anxiety disorder, phobic syndromes of all types, mania, manic depressive illness, hypomania, unipolar depression, depression, stress disorders, PTSD, somatoform disorders, personality disorders, psychosis, and schizophrenia), and drug dependence (e.g., alcohol, psychostimulants (eg, crack, cocaine, speed, meth), opioids, and nicotine), dementia-related conditions, such as epilepsy, seizure disorders, acute pain, chronic pain, chronic neuropathic pain may be treated using the combinations and methods described herein. The combinations of the invention are also useful for the treatment and prevention of other disorders including headaches (e.g., migraine, tension, and cluster), cerebrovascular disease, motor neuron diseases (e.g., ALS, Spinal motor atrophies, Tay-Sach's, Sandoff disease, familial spastic paraplegia), dementias (e.g., Alzheimer's disease, Parkinson's disease, Picks disease, fronto-temporal dementia, vascular dementia, normal pressure hydrocephalus, HD, and MCI), neurodegenerative diseases (e.g., familial Alzheimer's disease, prion-related diseases, cerebellar ataxia, Friedrich's ataxia, SCA, Wilson's disease, RP, ALS, Adrenoleukodystrophy, Menke's Sx, cerebral autosomal dominant arteriopathy with subcortical infarcts (CADASIL); spinal muscular atrophy, familial ALS, muscular dystrophies, Charcot Marie Tooth diseases, neurofibromatosis, von-Hippel Lindau, Frangile X, spastic paraplesia, Tuberous sclerosis, and Wardenburg syndrome), strokes (e.g, thrombotic, embolic, thromboembolic, hemmorhagic, venoconstrictive, and venous), movement disorders (e.g., PD, dystonias, benign essential tremor, tardive dystonia, tardive dyskinesia, and Tourette's syndrome), ataxic syndromes, disorders of the sympathetic nervous system (e.g., Shy Drager, Olivopontoicerebellar degeneration, striatonigral degenration, PD, HD, Gullian Barre, causalgia, complex regional pain syndrome types I and II, diabetic neuropathy, and alcoholic neuropathy), Cranial nerve disorders (e.g., Trigeminal neuropathy, trigeminal neuralgia, Menier's syndrome, glossopharangela neuralgia, dysphagia, dysphonia, and cranial nerve palsies), myelopethies, traumatic brain and spinal cord injury, radiation brian injury, multiple sclerosis, Post-menengitis syndrome, prion diseases, myelities, radiculitis, neuropathies (e.g., Guillian-Barre, diabetes associated with dysproteinemias, transthyretin-induced neuropathies, neuropathy associated with HIV, neuropathy associated with Lyme disease, neuropathy associated with herpes zoster, carpal tunnel syndrome, tarsal tunnel syndrome, amyloid-induced neuropathies, leprous neuropathy, Bell's palsy, compression neuropathies, sarcoidosis-induced neuropathy, polyneuritis cranialis, heavy metal induced neuropathy, transition metal-induced neuropathy, drug-induced neuropathy), pain syndromes (e.g., acute, chronic, neuropathic, nociceptive, central, and inflammatory), axonic brain damage, encephalopathies, and chronic fatigue syndrome. Any of these conditions may be treated using the methods and compositions described herein.

Treatment of a subject with the combination may be monitored using methods known in the art. The efficacy of treatment using the combination is preferably evaluated by examining the subject's symptoms in a quantitative way, e.g., by noting a decrease in the frequency of relapses, or an increase in the time for sustained worsening of symptoms. In a successful treatment, the subject's status will have improved (i.e., frequency of relapses will have decreased, or the time to sustained progression will have increased).

The invention will be illustrated in the following non-limiting examples.

### EXAMPLE 1

### In Vivo Method for Determining Optimal Steady-State Concentration Ratio (C_{ratio,ss})

A dose ranging study is performed in an appropriate depression model (e.g., forced swim test (FST)) with memantine to determine the ED50, which is approximately 15 μm. The ED50for the ADD (e.g., fluoxetine) is determined in a similar manner. An isobolic experiment ensues where the drugs are combined in fractions of their EDXXs to add up to ED100 (i.e., ED50:ED50, ED25:ED75, etc.). The plot of the data is constructed. The experiment points that lie below the straight line between the ED50 points on the graph are indicative of synergy, points on the line are indicative of additive effects, and points above the line are indicative of inhibitory effects. The point of maximum deviation from the isobolic line is the optimal ratio. This is the optimal steady state ratio (C_{ratio,ss}) and is adjusted based upon the components half-life. Similar protocols may be applied in a wide variety of validated animal models.

### EXAMPLE 2

### Combinations of an NMDA Receptor Antagonist and an ADD

Representative combination ranges and ratios are provided below for compositions of the invention. These ranges are based on the formulation strategies described herein.

Adult Dosage and Ratios for Combination Therapy

| | ADD Quantity, mg/day/(ADD:NMDA Ratio Range) | | | | | | |
|---|---|---|---|---|---|---|---|
| NMDA drug | Desipramine/ | Escitalopram/ | Paroxetine/ | Duloxetine/ | Venlafaxine/ | Buspirone/ | Bupropion/ |
| mg/day | NORPRAMIN ™ | LEXAPRO ™ | PAXIL ™ | CYMBALTA ™ | EFFEXOR ™ | BUSPAR ™ | WELLBUTRIN ™ |
| Memantine/ | 25-200 | 5-20 | 5-50 | 10-100 | 25-250 | 5-50 | 50-500 |
| 2.5-80 | (0.3-80) | (0.05-10) | (0.05-20) | (0.1-40) | (0.25-100) | (0.05-20) | (0.5-200) |
| Amantadine/ | 25-200 | 5-20 | 5-50 | 10-100 | 25-250 | 5-50 | 50-500 |
| 50-400 | (0.06-5) | (0.012-0.4) | (0.012-1 | (0.025-2) | (0.06-60) | (0.012-20) | (0.12-10) |
| Rimantadine/ | 25-200 | 5-20 | 5-50 | 10-100 | 25-250 | 5-50 | 50-500 |
| 50-200 | (0.3-80) | (0.05-10) | (0.05-20) | (0.1-40) | (0.25-100) | (0.05-20) | (0.5-200) |

### EXAMPLE 3

### Release Profile of Memantine and Paroxetine

Release proportions are shown in the tables below for a combination of memantine and paroxetine. The cumulative fraction is the amount of drug substance released from the formulation matrix to the serum or gut environment (e.g., U.S. Pat. No. 4,839,177).

| | MEMANTINE T½ = 60 hrs | PAROXETINE T½ = 21 hrs |
|---|---|---|
| Time | cum. fraction A | cum. fraction B |
| 1 | 0.2 | 0.2 |
| 2 | 0.3 | 0.3 |
| 4 | 0.4 | 0.4 |
| 8 | 0.5 | 0.5 |
| 12 | 0.6 | 0.6 |
| 16 | 0.7 | 0.7 |
| 20 | 0.8 | 0.8 |
| 24 | 0.9 | 0.9 |

### EXAMPLE 4

### Tablet Containing a Combination of Memantine and Venlafaxine

An extended release dosage form for administration of memantine and venlafaxine is prepared as three individual compartments. Three individual compressed tablets, each having a different release profile, followed by encapsulating the three tablets into a gelatin capsule and then closing and sealing the capsule. The components of the three tablets are as follows.

| Component | Function | Amount per tablet | |
|---|---|---|---|
| TABLET 1 (immediate release): | | | |
| Memantine | Active agent | 0 | mg |
| Venlafaxine | Active agent | 20 | mg |
| Dicalcium phosphate dihydrate | Diluent | 26.6 | mg |
| Microcrystalline cellulose | Diluent | 26.6 | mg |
| Sodium starch glycolate | Disintegrant | 1.2 | mg |
| Magnesium Stearate | Lubricant | 0.6 | mg |
| TABLET 2 (3-5 hour release): | | | |
| Memantine | Active agent | 10 | mg |
| Venlafaxine | Active agent | 40 | mg |
| Dicalcium phosphate dihydrate | Diluent | 26.6 | mg |
| Microcrystalline cellulose | Diluent | 26.6 | mg |
| Sodium starch glycolate | Disintegrant | 1.2 | mg |
| Magnesium Stearate | Lubricant | 0.6 | mg |
| Eudragit RS30D | Delayed release | 4.76 | mg |
| Talc | Coating component | 3.3 | mg |
| Triethyl citrate | Coating component | 0.95 | mg |
| TABLET 3 (Release delayed | | | |
| 7-10 hours): | | | |
| Memantine | Active agent | 12.5 | mg |
| Venlafaxine | Active agent | 60 | mg |
| Dicalcium phosphate dihydrate | Diluent | 26.6 | mg |
| Microcrystalline cellulose | Diluent | 26.6 | mg |
| Sodium starch glycolate | Disintegrant | 1.2 | mg |
| Magnesium Stearate | Lubricant | 0.6 | mg |
| Eudragit RS30D | Delayed release | 6.5 | mg |
| Talc | Coating component | 4.4 | mg |
| Triethyl citrate | Coating component | 1.27 | mg |

The tablets are prepared by wet granulation of the individual drug particles and other core components as may be done using a fluid-bed granulator, or are prepared by direct compression of the admixture of components. Tablet 1 is an immediate release dosage form, releasing the active agents within 1-2 hours following administration. It contains no memantine to avoid the dC/dT effects of the current dosage forms. Tablets 2 and 3 are coated with the delayed release coating material as may be carried out using conventional coating techniques such as spray-coating or the like. The specific components listed in the above tables may be replaced with other functionally equivalent components, e.g., diluents, binders, lubricants, fillers, coatings, and the like.

Oral administration of the capsule to a patient will result in a release profile having three phases, with initial release of the venlafaxine from the first tablet being substantially immediate, release of the memantine and venlafaxine from the second tablet occurring predominantly 3-5 hours following administration, and release of the memantine and venlafaxine from the third tablet occurring predominantly 7-9 hours following administration.

### EXAMPLE 5

### Beads Containing a Combination of Memantine and Venlafaxine

The method of Example 4 is repeated, except that drug-containing beads are used in place of tablets. A first fraction of beads is prepared by coating an inert support material such as lactose with the drug which provides the first (immediate release) pulse. A second fraction of beads is prepared by coating additional inert support material with a combination of the memantine and venlafaxine and coating these beads with an amount of enteric coating material sufficient to provide a drug release centering around 3-7 hours. A third fraction of beads is prepared by coating additional inert support material with a further combination of the memantine and venlafaxine and coating these with a greater amount of enteric coating material, sufficient to provide a drug release period centered around 7-12 hours. The three groups of beads may be encapsulated as in Example 4, or compressed, in the presence of a cushioning agent, into a single tablet. Alternatively, three groups of drug particles may be provided and coated as above, in lieu of the drug-coated lactose beads.

### EXAMPLE 6

### Release Profiles of IR and CR Escitalopram Formulations

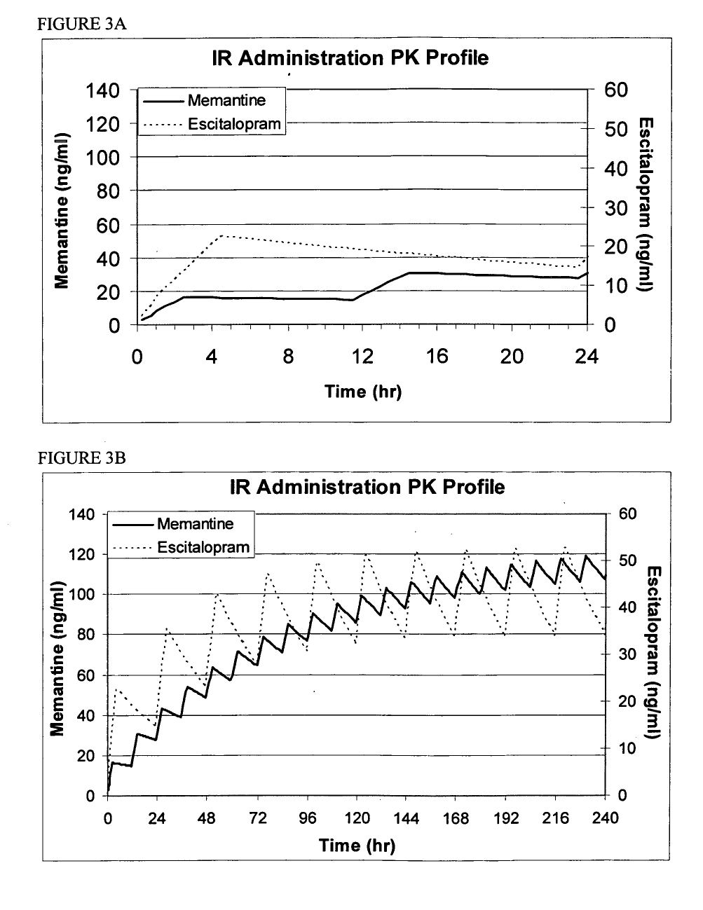

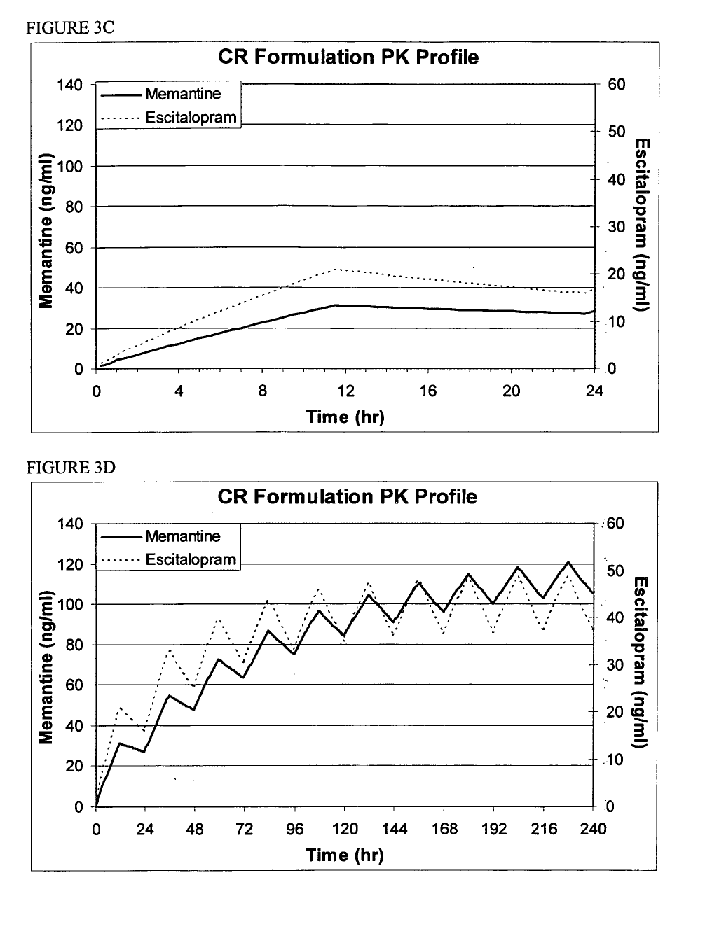

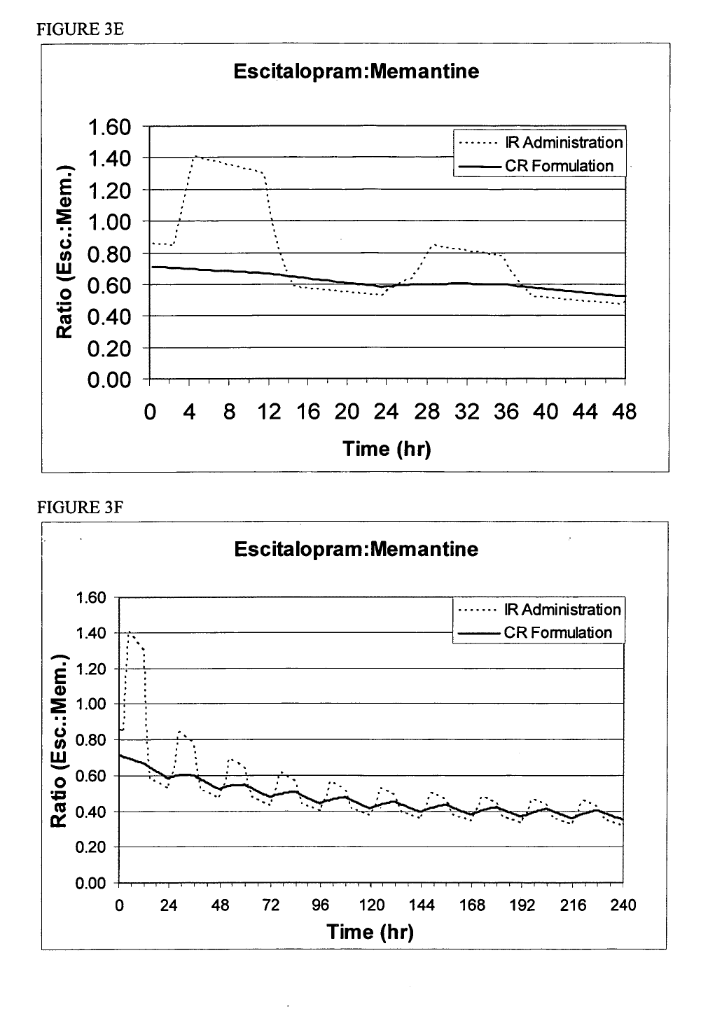

Exemplary human PK release profiles and Cratios are shown in FIGS. 3A-3F for a controlled release combination product made similar to Example 5. and compared to IR administration of presently marketed products. For the IR administration, oral dosing is 20 mg memantine b.i.d. and 20 mg escitalopram qd. For CR formulation 1, the 20 mg memantine and 20 mg escitalopram are provided in a controlled release oral delivery formulation releasing the active agents at a constant rate over twelve hours. This CR product will maintain nearly constant Cratios for the two active components, with Cratio.var calculated at 6% and 4% over time ranges from 2-24 hours and 192-240 hours.

In addition to achieving the desired release profile, this combination formulation will exhibit a preferred decrease in dC/dT and Cmax/Cmean, even with a higher dose of the NMDAr antagonist, thus the present invention may provide greater doses for increased therapeutic effect without escalation that might otherwise be required. Furthermore, the increased dosing allows less frequent administration of the therapeutic agents.

| | |
|---|---|
| IR (10 mg) | CR (20 mg) |

NMDAr Antag

| | | |
|---|---|---|
| dC/dT (4 hr) | 4.0 | 3.1 |
| Cmax/Cmean2 − 16 | 1.6 | 1.4 |

escitalopram

| | | |
|---|---|---|
| dC/dT (4 hr) | 5.1 | 2.1 |
| Cmax/Cmean2 − 16 | 1.2 | 1.4 |

### EXAMPLE 7

### A Patch Providing Extended Release of Memantine and Escitalopram

As described above, extended release formulations of an NMDA antagonist are formulated for topical administration. Memantine transdermal patch formulations are prepared as described, for example, in U.S. Pat. Nos. 6,770,295 and 6,746,689.

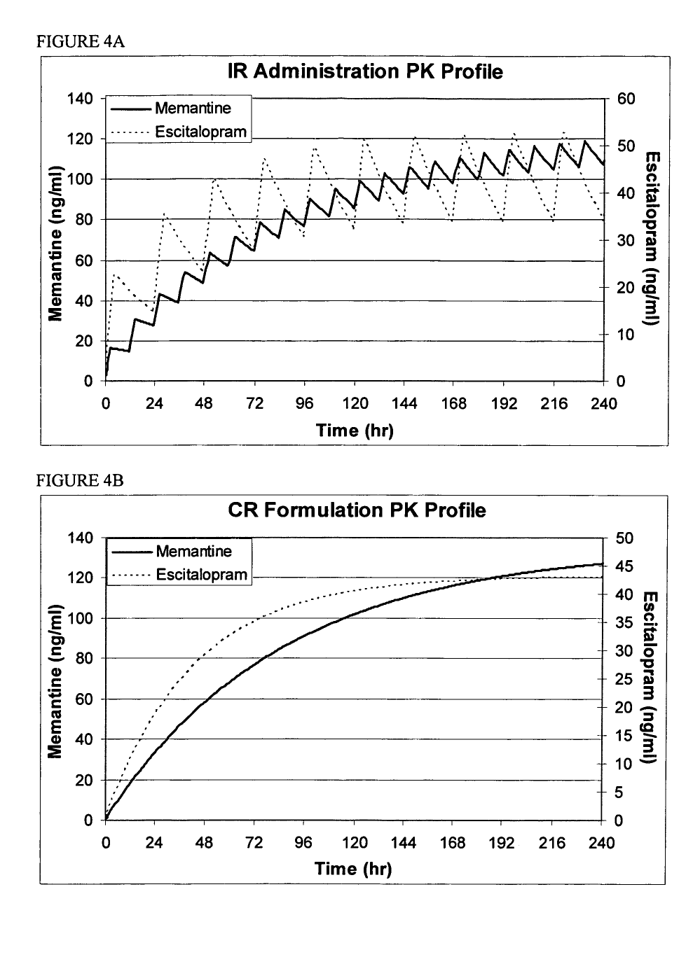

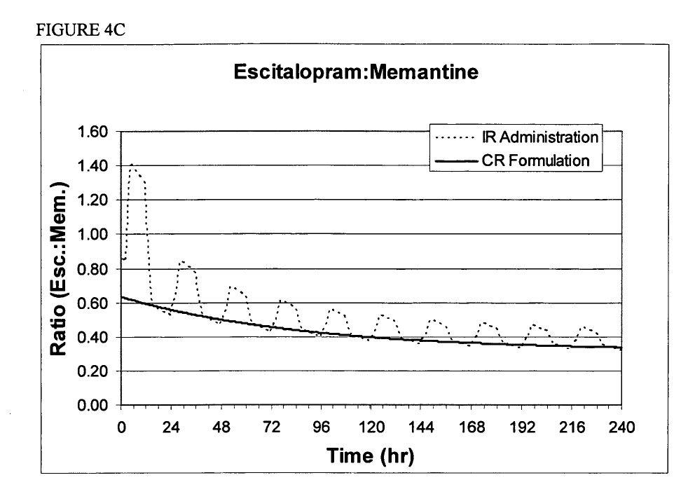

For the preparation of a drug-in-adhesive acrylate, 4.1 g of memantine and 3.6 g of escitalopram are dissolved in 11 g of ethanol and this mixture is added to 20 g of Durotak 387-2287 (National Starch & Chemical, U.S.A.). The drug gel is coated onto a backing membrane (Scotchpak 1012; 3M Corp., U.S.A.) using a coating equipment (e.g., RK Print Coat Instr. Ltd, Type KCC 202 control coater). The wet layer thickness is 400 μm. The laminate is dried for 20 minutes at room temperature and then for 30 minutes at 40° C. A polyester release liner is laminated onto the dried drug gel. The sheet is cut into patches and stored at 2-8° C. until use (packed in pouches). The concentration of memantine in the patches ranges between 4.6 and 6.6 mg/cm², while escitalopram ranges between 4.0 and 6.0 mg/cm². FIGS. 4A, 4B, and 4C are graphs comparing the anticipated immediate release profile with the anticipated 24 hour release of the current example. These graphs indicate the advantage of nearly continuous infusion of the components, and the importance of establishing the correct steady-state ratio (Cratio,ss) and then modifying the dosage form concentrations to achieve the optimal therapeutic effects.

Additional embodiments are within the claims.

1. A pharmaceutical composition comprising:
(a) an NMDA receptor antagonist;
(b) a second agent, wherein said agent is an anti-depressive drug (ADD); and
(c) a pharmaceutically acceptable carrier,
wherein at least one of said NMDA receptor antagonist or said second agent is provided in an extended release dosage form.

2. The pharmaceutical composition of claim 1 wherein said NMDA receptor antagonist has a dC/dT less than about 80% of the rate for the IR formulation.

3. The pharmaceutical composition of claim 1 wherein said NMDA receptor antagonist has a Cₘₐₓ/Cₘₑₐₙ of approximately 1.6 or less, approximately 2 hours to at least 12 hours after said NMDA receptor antagonist is introduced into a subject.

4. The pharmaceutical composition of claim 1, wherein the relative Cratio.var of said NMDA receptor antagonist and said second ADD is less than 100% from 2hour to 12 hours post administration.

5. The pharmaceutical composition of claim 1, wherein the relative Cratio.var of said NMDA receptor antagonist and said second ADD is less than 70% of the corresponding IR formulation from 2 hour to 12 hours post administration.

6. The pharmaceutical composition of claim 1, wherein said second agent is a selective serotonin re-uptake inhibitor (SSRI), a serotonin/norepinepherine reuptake inhibitors (SNRI) a tricyclic antidepressant (TCA).

7. The pharmaceutical composition of claim 1, wherein said NMDA receptor antagonist is memantine and said second agent is despramine, escitalopram, paroxetine, venlafaxine, duloxetine, buspirone, or bupropion.

8. The pharmaceutical composition of claim 1, wherein said pharmaceutical composition is formulated for oral, transnasal, parenteral, subtopical transepithelial, transdermal patch, subdermal, or inhalation delivery.

9. The pharmaceutical composition of claim 9, wherein said pharmaceutical composition is formulated as a suspension, capsule, tablet, suppository, lotion, or patch.

10. The pharmaceutical composition of claim 1, wherein said NMDA receptor antagonist is memantine and said second agent is duloxetine.

11. A method of treating a CNS-related condition comprising administering to a subject in need thereof a therapeutically effective amount of a combination comprising an NMDA receptor antagonist and a second agent, wherein said second agent is an AED, wherein said NMDA receptor antagonist is provided in an extended release dosage form.

12. The method of claim 11, wherein said CNS-related condition is epilepsy, seizure disorder, or convulsive disorder.

13. The method of claim 11, wherein said NMDA receptor antagonist and said second agent are administered simultaneously or sequentially.

14. The method of claim 11, wherein said NMDA antagonist and said second agent are administered as a single composition.

15. The method of claim 11, wherein said CNS-related condition is chronic nociceptive pain.

16. The method of claim 11, wherein said NMDA receptor antagonist is memantine and said second agent is duloxetine
